(19) United States
(12) Patent Application Publication
(10) Pub. No.: US 2017/0105924 A1
(43) Pub. Date: Apr. 20, 2017

(54) COMPOSITION

(71) Applicant: COSMETIC WARRIORS LIMITED, Poole, Dorset (GB)

(72) Inventors: Mark CONSTANTINE, Poole, Dorset (GB); Margaret Joan CONSTANTINE, Poole, Dorset (GB); Helen Elizabeth AMBROSEN, Wimborne, Dorset (GB); Rowena Jacqueline BIRD, Christchurch, Dorset (GB)

(21) Appl. No.: 15/317,836

(22) PCT Filed: Jun. 12, 2015
(86) PCT No.: PCT/GB2015/051729

§ 371 Date: Dec. 9, 2016

(30) Foreign Application Priority Data
Jun. 12, 2014 (GB) 1410439.9

(51) Int. Cl. A61K 8/92; A61K 8/40; A61Q 17/04; A61K 8/26; A61K 8/27; A61K 8/02; A61K 8/37; A61K 8/35

(52) U.S. Cl. CPC A61K 8/922; A61K 8/37; A61K 8/40; A61K 8/35; A61K 8/26; A61K 8/27; A61K 8/0216; A61Q 17/04; A61K 2800/31

(57) ABSTRACT

A sunscreen in solid form includes (i) vegetable oil, vegetable butter or mixture thereof, vegetable oil and vegetable butter are present in a total amount of from 40 to 80% by weight of the solid sunscreen, (ii) one or more UV absorbing materials, the UV absorbing material is present in an amount of from 10 to 55% by weight of the solid sunscreen, and (iii) one or more UV reflecting materials, the UV reflecting material is present in an amount of from 0.5 to 30% by weight of the solid sunscreen.

### FIELD OF THE INVENTION

The present invention relates to a sunscreen, a process for producing said sunscreen, and a product prepared by the method.

### BACKGROUND TO THE INVENTION

The present invention relates to products particularly those for use in contact with the human body.

Sunscreens are any type of topical product that can absorb or reflect Ultraviolet A (UVA) or Ultraviolet B (UVB) radiation from sunlight, and therefore help protect the skin against sunburn and other potential harmful effect of the sun. Excessive UV radiation exposure is the leading cause of primarily non-malignant skin tumours.

The first sunscreen was reported to have been invented by Franz Greiter in 1938; the product ‘Glacier Cream’ became the basis for Piz Buin company, who took their name from the area in Switzerland that inspired Greiter's cream. Greiter in 1962 also developed the concept of a Sun Protection Factor, which has become the worldwide standard for measuring the efficacy of sunscreens when applied at an even rate of 2 milligrams per square centimetre (mg/cm²).

The Ultraviolet radiation has different properties dependant on its wavelength. The UV spectra can be split into 3 types, as discussed below:

1. UVA radiation penetrates the skin more deeply than UVB or UVC (which is blocked by the Ozone layer) reaching the dermis through the epidermis. The effect of UVA exposure on the skin includes pigment darkening, photo aging and indirect DNA damage which can lead to the formation of cancerous cells.

Protection from UVA radiation is measured using 2 standard tests—Persistent Pigment Darkening and an In-Vitro UVA test. In Japan products receive either a PA+, PA++ or PA+++ rating depending on their efficacy. In Europe, the COLIPA UVA symbol can be applied to products whose UVA Protection Factor is at least ⅓ of the SPF value.

2. UVB mostly penetrates the skin only as far as the epidermis and exposure only occurs outdoors. The effect of UVB radiation includes sunburn, delayed tan, skin thickening and direct DNA damage. UVB protection is investigated using the Sun Protection Factor test. The SPF is derived from the smallest dose causing an erythema (reddening of the skin) with sunscreen compared to without sunscreen.

3. UVC, which doesn't reach the earth, as it is blocked by the ozone layer.

There are two types of sunscreen agents, UV reflectors (Physical Sunscreens) and UV absorbers (Chemical Sunscreens).

1. UV reflectors or physical sunscreens protect the skin from the sun by deflecting or blocking the sun's rays. Titanium Dioxide and Zinc Oxide are the most commonly used physical sunscreens, as they are inorganic particles they cannot be absorbed by the body. Therefore they can form a filter on the skin which reflects or scatters UV radiation. Zinc Oxide is the only true UV reflector as Titanium Dioxide has both reflector and absorber properties. UV reflectors by nature tend to be thicker than UV reflectors and are often harder to apply. They also tend to leave the skin white or tinted. In addition, as they are a physical medium they tend to rub off more easily and therefore have to be reapplied more frequently.

2. UV absorbers or Chemical Sunscreens absorb the UVA and UVB radiation and convert them into harmless heat. An important feature is that the absorber molecules convert the energy without reacting with other molecules or being destroyed. These filters respond to the UV radiation by undergoing a short-term internal rearrangement reaction known as a H shift. The absorbed UV radiation stimulates the molecule and one hydrogen atom migrates to a different position inside the molecule. When converted back into its original state, the energy absorbed as UV light is gradually released as heat, allowing the molecule to absorb further radiation. This mechanism ensures effective and long-lasting protection. They tend to be colourless, odourless and runny which makes them the ideal ingredients for sunscreen creams or lotions.

Physical sunscreens tend to be better tolerated by most skin types. However, they tend to leave a white cast or white streaks after application and don't offer as much UVA protection compared to chemical sunscreens. Physical sunscreens are also a bit thicker so they are often more difficult to apply. Since reflectors and absorbers have their advantages and disadvantages, many of today's sunscreens contain both UV filters. However, knowing whether a sunscreen is physical, chemical, or both does not tell you enough information about whether a particular sunscreen will be a protective one,

The general public have a perfunctory attitude to cosmetic lotions that provide protection from UVA and UVB radiation from sunlight. Consumers only apply sunscreen lotions when the weather conditions obviously demand it and the lotions within this field offer limited functionality when it comes to the considering the various requirements from the general public. This can often lead to the consumer either using the incorrect product, in the incorrect manner or potentially not protecting themselves at all from the harmful effects of the sun.

The present invention seeks to provide a sunscreen that addresses the problems of the prior art.

### SUMMARY OF THE INVENTION

The present invention relates to a sunscreen that can be readily washed on by the user. The sunscreen is in the form of a predefined solid product which can be washed on while the user is bathing, or after bathing by the user when skin is wet and then washed off, leaving the skin protected from UV radiation.

In a first aspect, there is provided a sunscreen in solid form comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a second aspect, there is provided a sunscreen in solid form, formulated such that the sunscreen may be washed on to the body.

In a third aspect, there is provided a sunscreen in solid form, formulated such that when a predefined dose of the sunscreen is applied to the body, adequate coverage of the body to a specified UV protective rating is provided.

In a fourth aspect, there is provided a process for the production of a sunscreen in solid form comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen,
   the process comprising the steps of
(a) when the vegetable oil, vegetable butter or mixture thereof is solid, heating the vegetable oil, vegetable butter or mixture thereof to provide a liquid
(b) mixing the one or more UV absorbing materials, and the one or more UV reflecting material, with the liquid vegetable oil, vegetable butter or mixture thereof
(c) cooling the mixture to provide the sunscreen in a predefined solid shape.

In a fifth aspect, there is provided use of a sunscreen for reducing the damage to skin of a user by sunlight, wherein the sunscreen is in solid form and comprises
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a sixth aspect, there is provided a method of washing on a sunscreen comprising the step(s) of applying to the wet skin of a user a sunscreen in solid form and comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

The nature of the solid sunscreen is such that a particular dose of material may be used by the user and washed on to the body. The dose of solid product is washed on in its entirety, so the user can have confidence that the appropriate amount of product has been applied and that the required sunscreen effect will be achieved. Thus the problems of the prior art when lotion type materials are applied to the body until the user “feels” that full coverage has been achieved are overcome. Furthermore, by applying the solid sunscreen to the body during bathing, or showering, the user is able to apply the sunscreen to all parts of the body while in private.

This addresses the problems of the lotions of the prior art which leave an oily residue or sticking feeling which discourages users from applying the product before dressing. Once the user is dressed, the application of sunscreen is a greater inconvenience and also risks contacting the lotion with the user's clothes resulting in staining etc. Most importantly the user may not apply enough sunscreen lotion to provide adequate protection, thus the present invention provides a sunscreen which is more likely to be applied by the user and, when washed on, will be applied to greater area of the user's skin, and can be correctly dosed by the user, therefore resulting in greater sun protection.

For ease of reference, these and further aspects of the present invention are now discussed under appropriate section headings. However, the teachings under each section are not necessarily limited to each particular section.

### DETAILED DESCRIPTION

### Composition

As discussed herein, in one aspect of the present invention, there is provided a sunscreen in solid form comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

As discussed herein, the sunscreen is a solid. Solid products of the present invention are compositions which can substantially sustain their physical shape when unsupported by external means, e.g. packaging etc. Thus, they are considered to be solid, solid like, in solid form or in solid-like form at room temperature. For the avoidance of doubt the solid product must remain substantially solid at up to 30° C.

By solid-like, it is understood that some materials are considered to be solid, yet over an extremely long period of time, may alter in shape, e.g. amorphous materials such as glass etc. However, they are considered to be solid-like as, for the purpose they fulfil, they are solid. The solid form of the solid compositions of the present invention means that external packaging is not required to maintain the shape of the composition.

Vegetable Oil, Vegetable Butter or Mixtures thereof

The sunscreen of the present invention may contain vegetable oil, vegetable butter or mixture thereof in a total amount of from 40 to 80% by weight of the sunscreen.

In one aspect the sunscreen comprises vegetable oil, vegetable butter or mixture thereof in a total amount of from 40 to 75% by weight of the sunscreen, such as in a total amount of from 40 to 70% by weight of the sunscreen, such as in a total amount of from 40 to 65% by weight of the sunscreen, such as in a total amount of from 40 to 60% by weight of the sunscreen, such as in a total amount of from 45 to 55% by weight of the sunscreen, such as in a total amount of approximately 50% by weight of the sunscreen.

In one aspect the sunscreen comprises vegetable oil, vegetable butter or mixture thereof in a total amount of from 45 to 80% by weight of the sunscreen, such as in a total amount of from 50 to 80% by weight of the sunscreen, such as in a total amount of from 55 to 80% by weight of the sunscreen, such as in a total amount of from 60 to 80% by weight of the sunscreen, such as in a total amount of from 60 to 75% by weight of the sunscreen, such as in a total amount of from 60 to 70% by weight of the sunscreen.

The vegetable oil and vegetable butter may be selected from any materials suitable to achieve the purpose of the present invention. In one preferred aspect, the vegetable oil and vegetable butter are selected from cocoa butter, murumuru butter, cupuacu butter, illipe butter, mango butter, sesame oil, rosehip oil, almond oil, raspberry seed oil, and mixtures thereof. In one preferred aspect, the vegetable oil and vegetable butter are selected from avocado butter, shea butter, cocoa butter, illipe butter, mango butter, rosehip oil, almond oil, moringa oil, and mixtures thereof.

### UV Absorbing Material

The sunscreen of the present invention contains a UV absorbing material. As discussed herein, UV absorbers absorb the UVA and/or UVB radiation and convert them into harmless heat. An important feature is that the absorber molecules convert the energy without reacting with other molecules or being destroyed. These filters respond to the UV energy by undergoing a short-term internal rearrangement When converted back into its original state, the energy absorbed as UV light is gradually released as heat, allowing the molecule to absorb further radiation.

The UV absorbing material of the present invention is a material that absorbs at least UVA or UVB radiation. In one aspect, the UV absorbing material absorbs both UVA and UVB radiation. It will be appreciated by one skilled in the art that the present invention encompasses aspects where the sunscreen composition contains one material which absorbs UVA radiation and another material which absorbs UVB radiation. By UV absorbing it is meant a material that absorbs radiation in the ultraviolet range at wavelengths of 290 to 400 nanometres.

Preferably the sunscreen comprises UV absorbing material in an amount of from 10 to 50% by weight of the sunscreen, such as in an amount of from 10 to 45% by weight of the sunscreen, such as in an amount of from 10 to 40% by weight of the sunscreen, such as in an amount of from 10 to 35% by weight of the sunscreen, such as in an amount of from 10 to 30% by weight of the sunscreen, such as in an amount of from 10 to 25% by weight of the sunscreen, such as in an amount of from 10 to 20% by weight of the sunscreen, such as in an amount of from 15 to 25% by weight of the sunscreen, such as in an amount of from 15 to 20% by weight of the sunscreen, such as in an amount of approximately 18% by weight of the sunscreen.

The UV absorbing material may be selected from any materials suitable to achieve the purpose of the present invention. in one preferred aspect, the UV absorbing material is selected from octocrylene, octyl methoxycinnamate, butylmethoxydibenzoylmethane, homosalate, ecamsule, and mixtures thereof. In one preferred aspect, the UV absorbing material is selected from octocrylene, butylmethoxydibenzoylmethane, homosalate and mixtures thereof.

### UV Reflecting Material

The sunscreen of the present invention contains a UV reflecting material. As discussed herein, UV reflecting materials protect the skin from the sun by deflecting or blocking the sun's rays.

The UV reflecting material of the present invention is material that reflects at least UVA or UVB radiation. In one aspect, the UV reflecting material absorbs both UVA and UVB radiation. It will be appreciated by one skilled in the art that the present invention encompasses aspects where the sunscreen composition contains one material which reflects UVA radiation and another material which reflects UVB radiation. By UV reflecting it is meant a material that reflects or scatters radiation in the ultraviolet range at wavelengths of 290 to 400 nanometres.

Preferably the sunscreen comprises UV reflecting material in an amount of from 1 to 30% by weight of the sunscreen, such as in an amount of from 5 to 30% by weight of the sunscreen, such as in an amount of from 5 to 25% by weight of the sunscreen, such as in an amount of from 8 to 25% by weight of the sunscreen, such as in an amount of from 8 to 23% by weight of the sunscreen, such as in an amount of from 10 to 25% by weight of the sunscreen, such as in an amount of from 15 to 25% by weight of the sunscreen, such as in an amount of from 15 to 20, such as in an amount of approximately 18% by weight of the sunscreen.

The UV reflecting material may be selected from any materials suitable to achieve the purpose of the present invention. In one preferred aspect, the UV reflecting material is selected from clays, zinc oxide, titanium dioxide and mixtures thereof. In one preferred aspect, the UV reflecting material is selected from calamine kaolin, zinc oxide, titanium dioxide and mixtures thereof. In one preferred aspect, the UV reflecting material is selected from calamine kaolin, zinc oxide and mixtures thereof.

One preferred aspect, the UV reflecting material comprises at least a clay. In this aspect, the claim may be used alone or in combination with other materials. For example, the UV reflecting material may comprise at least (i) a clay and (ii) at least one of zinc oxide and titanium dioxide.

The clay may be selected from suitable known clays, which may act as UV reflective materials. In one aspect, the clay is selected from calamine, kaolin, talc, rhassoul mud powder, mica and mixtures thereof.

Calamine is a mixture of zinc oxide (ZnO) with 0.5% of either ferric oxide (Fe₂O₃) or a zinc carbonate.

In one preferred aspect, the UV reflecting material is at least zinc oxide. In one preferred aspect, the UV reflecting material is selected from calamine, kaolin and mixtures thereof.

Preferably the sunscreen comprises zinc oxide in an amount of from 1 to 30% by weight of the sunscreen, such as in an amount of from 5 to 30% by weight of the sunscreen, such as in an amount of from 5 to 25% by weight of the sunscreen, such as in an amount of from 8 to 25% by weight of the sunscreen, such as in an amount of from 8 to 23% by weight of the sunscreen, such as in an amount of from 10 to 25% by weight of the sunscreen, such as in an amount of from 15 to 25% by weight of the sunscreen, such as in an amount of from 15 to 20

Preferably the sunscreen comprises calamine in an amount of from 1 to 30% by weight of the sunscreen, such as in an amount of from 5 to 30% by weight of the sunscreen, such as in an amount of from 5 to 25% by weight of the sunscreen, such as in an amount of from 8 to 25% by weight of the sunscreen, such as in an amount of from 8 to 23% by weight of the sunscreen, such as in an amount of from 10 to 25% by weight of the sunscreen, such as in an amount of from 15 to 25% by weight of the sunscreen, such as in an amount of from 15 to 20

It will be appreciated by one skilled in the art that the nature of UV reflecting materials are such that they may reflect other wavelengths of the visible spectrum. In this respect, the UV reflecting material may also act as a pigment. The sunscreen may contain pigments in addition to the essential components recited herein. If the pigment is a UV reflecting material then the amount of pigment is to be incorporated in the total of UV reflecting material defined herein. Thus in one aspect the sunscreen may further comprise a pigment. The pigment may or may not be a UV reflecting material.

### UV Absorbing and UV Reflecting Materials

It will be appreciated by one skilled in the art that the UV absorbing and UV reflecting materials may have to be selected from materials which have undergone regulatory approval. In one aspect the UV absorbing and UV reflecting materials are selected from materials approved in Food and Drug Administration, 21 CFR Parts 310, 352, 700, and 740. In one aspect the UV absorbing and UV reflecting materials are selected from materials approved in Food and Drug Administration, 21 CFR Parts 310, 352, 700, and 740 as published in Federal Register Not. 64, No. 98/Friday, May 21, 1999/Rules and Regulations.

In one aspect the UV absorbing and UV reflecting materials are selected from the group consisting of aminobenzoic acid (PABA), avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide and mixtures thereof.

### Dosage Form

As discussed herein, a key advantage of the present invention is that the user applies a dose of solid product in its entirety, so the user can have confidence that the appropriate amount of product has been applied and that the required sunscreen effect will be achieved. Thus the problems of the prior art, when lotion type materials are applied to the body until the user “feels” that full coverage has been achieved, are overcome.

Preferably the solid sunscreen may be formed into a shape which allows for easy dosage of the product. In one aspect the product is formed into a single dosage unit which should be applied to achieve a determined SPF. The SPF is typically SPF 30. In one aspect the product is formed into a shape which allows for a single dosage to be removed from an overall larger piece of solid. As a non-limiting example, a bar may be provided with indentations or grooves, which allow for a dosage unit to be ‘snapped’ from the larger bar.

In one aspect the present invention provides a dosage form of a sunscreen, wherein the sunscreen is in solid form and comprises
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen,
   wherein the dosage form, when washed on to the body of a 70 kg human, is absorbed to provide a Sun Protection Factor (SPF) of at least 30. SPF is to be determined in accordance with ISO 24444. SPF may be determined in accordance with the method of Annex B.

### Further Components

The sunscreen may contain one or more additional components such as to provide the desired composition. In one aspect the solid sunscreen further comprising at least one additional component selected from waxes, humectants, surfactants, fruits, vegetables, herbs, seaweeds, cereals, beans, proteins, binders, fillers, dispersants, opacifiers, perfumes, colours, fragrances and mixtures thereof. In one aspect the solid sunscreen further comprising at least one additional component selected from humectants, surfactants, fruits, vegetables, herbs, seaweeds, cereals, beans, proteins, binders, fillers, dispersants, opacifiers, perfumes, colours, fragrances and mixtures thereof. In one aspect, the sunscreen comprising a further component selected from waxes, binders, fillers, fruits, vegetables, humectants, dispersants and mixtures thereof. In one aspect, the sunscreen comprising a further component selected from binders, fillers, fruits, vegetables, humectants, dispersants and mixtures thereof.

In one aspect the sunscreen contains one or more waxes. In one aspect the sunscreen comprises waxes in an amount of no greater than 30% by weight of the sunscreen. If present, waxes may be present in an amount of from 0.1 to 30% by weight of the total composition. The amount of fragrance may be from 1 to 30% by weight of the total composition, such as from 5 to 30% by weight of the total composition, such as from 10 to 30% by weight of the total composition, such as from 15 to 30% by weight of the total composition, such as from 15 to 25% by weight of the total composition, such as from 15 to 20% by weight of the total composition. Alternatively, in one aspect, the sunscreen is substantially free of waxes. In one aspect the sunscreen comprises waxes in an amount of no greater than 15% by weight of the sunscreen, such as in an amount of no greater than 10% by weight of the sunscreen, such as in an amount of no greater than 5% by weight of the sunscreen, such as in an amount of no greater than 2% by weight of the sunscreen, such as in an amount of no greater than 1% by weight of the sunscreen, such as in an amount of no greater than 0.5% by weight of the sunscreen, such as in an amount of no greater than 0.1% by weight of the sunscreen, such as in an amount of no greater than 0.01% by weight of the sunscreen.

If present, waxes may be selected from any materials suitable to achieve the purpose of the present invention. In one aspect, the wax is beeswax, rapeseed wax, japan wax and mixtures thereof.

In one aspect the sunscreen contains one or more fragrances. Preferably the sunscreen comprises fragrance in an amount of no greater than 5% by weight of the sunscreen. If present, fragrance may be present in an amount of from 0.1 to 5% by weight of the total composition. The amount of fragrance is preferably from 0.1% to 5% by weight of the total composition, such as from 0.1% to 4% by weight of the total composition, such as from 0.5% to 5% by weight of the total composition, such as from 1% to 5% by weight of the total composition, such as from 0.5% to 4% by weight of the total composition, such as from 0.5% to 3% by weight of the total composition, such as from 0.5% to 2.5% by weight of the total composition, such as from 1.5% to 2.5% by weight of the total composition. Alternatively, in one aspect, the sunscreen is fragrance free.

The essential oils may be selected based on the fragrance desired, skin type to be treated and other effects desired based on the well-known properties of essential oils. The addition of essential oils, when taken in to the nose, is known to alter mood. For example, essential oils are known to create effects of drowsiness or stimulating the senses. Many effects can be achieved by the use of essential oils.

In one embodiment, the one or more essential oils present in the product are selected from Rosewood, Sandalwood, Chamomile, Eucalyptus, Tonka absolute, Lemon myrtle, Jasmin, Ylang ylang, Labdanum, Lemongrass, Rose Absolute, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the product are selected from Rosewood, Sandalwood, Chamomile, Eucalyptus, Lavender, Tonka absolute, Rose absolute.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the product of a material, such as a natural material, that has a high vitamin content.

In one aspect the sunscreen further comprises a colorant material. If present, colorant materials may be present in an amount of from 0.001 to 3% by weight of the total composition.

In one aspect the sunscreen contains a humectant. In one aspect, the humectant is selected from honey, glycerine, sorbitol, agave nectar, fruit syrups, herbal syrups and mixtures thereof. Preferably the humectant is selected from honey, glycerine, sorbitol and mixtures thereof.

In one aspect the sunscreen further comprises a filler. The filler may be selected from flours, such as cornflour, and mixtures of flours.

In one aspect the solid sunscreen contains a dispersant. The dispersant is present to assist with the dispersion of the UV reflecting agent, such as a clay, in the oils, fats, waxes or mixtures thereof. In one aspect, the dispersant may be magnesium carbonate. In one aspect the sunscreen contains a dispersant in an amount of from 0.1 to 20% by weight of the total composition. The amount of dispersant is preferably from 1% to 20% by weight of the total composition, such as from 1% to 15% by weight of the total composition, such as from 2% to 15% by weight of the total composition, such as from 4% to 15% by weight of the total composition, such as from 4% to 12% by weight of the total composition, such as from 4.5% to 12% by weight of the total composition. In one aspect the sunscreen contains magnesium carbonate in an amount of from 0.1 to 20% by weight of the total composition. The amount of magnesium carbonate is preferably from 1% to 20% by weight of the total composition, such as from 1% to 15% by weight of the total composition, such as from 2% to 15% by weight of the total composition, such as from 4% to 15% by weight of the total composition, such as from 4% to 12% by weight of the total composition, such as from 4.5% to 12% by weight of the total composition.

In one aspect the sunscreen contains water. In one aspect the sunscreen is free or substantially free of water. Preferably the sunscreen comprises water in an amount of no greater than 5% by weight of the sunscreen, such as in an amount of no greater than 4% by weight of the sunscreen, such as in an amount of no greater than 3% by weight of the sunscreen, such as in an amount of no greater than 2% by weight of the sunscreen, such as in an amount of no greater than 1% by weight of the sunscreen, such as in an amount of no greater than 0.5% by weight of the sunscreen, such as in an amount of no greater than 0.1% by weight of the sunscreen, such as in an amount of no greater than 0.01% by weight of the sunscreen.

In one aspect the sunscreen contains preservatives. In one aspect the sunscreen is free or substantially free of preservatives. Preferably the sunscreen comprises preservatives in an amount of no greater than 5% by weight of the sunscreen, such as in an amount of no greater than 4% by weight of the sunscreen, such as in an amount of no greater than 3% by weight of the sunscreen, such as in an amount of no greater than 2% by weight of the sunscreen, such as in an amount of no greater than 1% by weight of the sunscreen, such as in an amount of no greater than 0.5% by weight of the sunscreen, such as in an amount of no greater than 0.1% by weight of the sunscreen, such as in an amount of no greater than 0.01% by weight of the sunscreen.

It will be appreciated by one skilled in the art that when the sunscreen is in the form of lotion it is typically provided to the end user contained within a packaging. However the advantage of the solid sunscreen of the present invention is that it does not require packaging. Thus preferably the sunscreen is provided free of packaging.

### Method

As discussed herein, in one aspect the present invention provides a method of applying a sunscreen comprising the step(s) of washing on to the wet skin of a user a sunscreen in solid form and comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

The method of applying the sunscreen is typically performed by the user applying the sunscreen during or immediately after bathing or showering. In a preferred aspect the sunscreen is washed on during bathing or showering and is absorbed by the skin. Any residue is washed off and the user pats dry, leaving the user with a feeling of washed and softened skin and importantly protected against UV radiation, rather than the greasy feeling of prior sunscreens.

### Further Aspects

In a further aspect, there is provided a sunscreen in solid form comprising
(i) vegetable oil, vegetable butter, wax or mixture thereof, wherein vegetable oil, vegetable butter and wax are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a further aspect, there is provided a process for the production of a sunscreen in solid form comprising
(i) vegetable oil, vegetable butter, wax or mixture thereof, wherein vegetable oil, vegetable butter and wax are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen,
   the process comprising the steps of
(a) when the vegetable oil, vegetable butter, wax or mixture thereof is solid, heating the vegetable oil, vegetable butter, wax or mixture thereof to provide a liquid
(b) mixing the one or more UV absorbing materials, and the one or more UV reflecting material, with the liquid vegetable oil, vegetable butter, wax or mixture thereof
(c) cooling the mixture to provide the sunscreen in a predefined solid shape.

In a further aspect, there is provided use of a sunscreen for reducing the damage to skin of a user by sunlight, wherein the sunscreen is in solid form and comprises
(i) vegetable oil, vegetable butter, wax or mixture thereof, wherein vegetable oil, vegetable butter and wax are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a further aspect, there is provided a method of washing on a sunscreen comprising the step(s) of applying to the wet skin of a user a sunscreen in solid form and comprising
(i) vegetable oil, vegetable butter, wax or mixture thereof, wherein vegetable oil, vegetable butter and wax are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In the above further aspects the sunscreen contains vegetable oil, vegetable butter, wax or mixture thereof in a total amount of from 40 to 80% by weight of the sunscreen. Preferably the sunscreen comprises vegetable oil, vegetable butter, wax or mixture thereof in a total amount of from 45 to 80% by weight of the sunscreen, such as in a total amount of from 50 to 80% by weight of the sunscreen, such as in a total amount of from 55 to 80% by weight of the sunscreen, such as in a total amount of from 60 to 80% by weight of the sunscreen, such as in a total amount of from 65 to 75% by weight of the sunscreen, such as in a total amount of from 40 to 75% by weight of the sunscreen, such as in a total amount of from 40 to 70% by weight of the sunscreen, such as in a total amount of from 40 to 65% by weight of the sunscreen, such as in a total amount of from 40 to 60% by weight of the sunscreen, such as in a total amount of from 45 to 55% by weight of the sunscreen, such as in a total amount of approximately 50% by weight of the sunscreen.

In the above further aspects the vegetable oil, vegetable butter and wax may be selected from any materials suitable to achieve the purpose of the present invention. In one preferred aspect, the vegetable oil, vegetable butter and wax are selected from cocoa butter, murumuru butter, cupuacu butter, illipe butter, mango butter, sesame oil, rosehip oil, almond oil, raspberry seed oil, beeswax, rapeseed wax, japan wax and mixtures thereof.

In a further aspect, there is provided a sunscreen in solid form comprising
(i) vegetable oil, vegetable butter or mixture thereof, wherein vegetable oil and vegetable butter are present in a total amount of from 30 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a further aspect, there is provided a process for the production of a sunscreen in solid form comprising
(i) vegetable oil, vegetable butter or mixture thereof, wherein vegetable oil and vegetable butter are present in a total amount of from 30 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen,
   the process comprising the steps of
(a) when the vegetable oil, vegetable butter or mixture thereof is solid, heating the vegetable oil, vegetable butter or mixture thereof to provide a liquid
(b) mixing the one or more UV absorbing materials, and the one or more UV reflecting material, with the liquid vegetable oil, vegetable butter or mixture thereof
(c) cooling the mixture to provide the sunscreen in a predefined solid shape.

In a further aspect, there is provided use of a sunscreen for reducing the damage to skin of a user by sunlight, wherein the sunscreen is in solid form and comprises
(i) vegetable oil, vegetable butter or mixture thereof, wherein vegetable oil and vegetable butter are present in a total amount of from 30 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

In a further aspect, there is provided a method of washing on a sunscreen comprising the step(s) of applying to the wet skin of a user a sunscreen in solid form and comprising
(i) vegetable oil, vegetable butter or mixture thereof, wherein vegetable oil and vegetable butter are present in a total amount of from 30 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

### EXAMPLES

The invention will now be described with reference to the following non-limiting example.

### Example 1

The following composition was prepared in accordance with the method below

| Phase | Formula % | Raw Material Type | Batch Size: g |
|---|---|---|---|
| A | 20.0 | Mango Butter | 200.000 |
| | 20.0 | Rapeseed Wax | 200.000 |
| | 10.0 | Rosehip Oil | 100.000 |
| | 8.0 | Homosalate | 80.000 |
| | 8.0 | Octocrylene | 80.000 |
| | 2.0 | Butylmethoxydibenzoylmethane | 20.000 |
| B | 8.0 | Kaolin | 80.000 |
| | 10.0 | Calamine Powder | 100.000 |
| | 12.0 | Magnesium Carbonate | 120.000 |
| C | 2.0 | Fragrance | 20.000 |
| | 100.000 | | 1000.000 |

1. The oil phase (A) (including any oil miscible materials such as sunscreens, waxes and other materials) is gradually warmed to between 50-95° C., with the ideal temperature being at 68° C.
2.The oil phase (A) is then be cooled to allow the formation of a specific crystalline structure (known as Form V), which allows the butters (once solidified) to melt in a more pleasant manner and at a temperature closer to body temperature, this is achieved at temperature between 25-32° C., with the ideal temperature being 30° C.
3. Once the butters have reached this temperature they must be reheated to prevent the formation of alternative crystalline structure which have lower melting temperature and cause the solid cosmetic composition melt at temperatures lower than body temperature. This also slows down the crystal growth so that the structure that forms is not too solid, i.e. won't melt on skin contact. This is achieved at temperatures of 33-45° C., with the ideal temperature being 36° C.
4. All the clays and powder based materials (B) are then sieved and ground together.
5. Phase A can be gradually added to B, mixing carefully in order to disperse the B effectively within A. This is achieved with phase A at a temperature of between 25-35° C., with an ideal temperature of 30° C.
6. The fragrance materials and any other materials can then be gradually stirred into AB, in order not to disrupt the crystal formation.
7. The composition can then be poured into moulds and left to completely solidify, at a temperature between 4-20° C., with the ideal temperature being 12° C.

The sunscreen was tested for its UVA “blocking” properties. The UVA value is ⅓ of the SPF value. The solid sunscreen products qualify for the UVA symbol, authorised by COLIPA. The results of this trial are provided in Annex A.

The sunscreen was tested to determine its Sun Protection Factor (SPF). The solid sunscreen products achieved an SPF value of 30. The results of this trial are provided in Annex B.

### Example 2

The following composition was prepared in accordance with the method of Example 1.

| | Formula % | Raw Material Type | |
|---|---|---|---|
| A | 40.0 | Cocoa Butter | 400.000 |
| | 15.0 | Avocado Butter | 150.000 |
| | 15.0 | Almond Oil | 150.000 |
| | 5.0 | Homosalate | 50.000 |
| | 4.0 | Octocrylene | 40.000 |
| | 2.0 | Butylmethoxydibenzoylmethane | 20.000 |
| B | 8.25 | Kaolin | 82.500 |
| | 9.75 | Magnesium Carbonate | 97.500 |
| C | 1.00 | Fragrance | 10.000 |
| | 100.000 | | 1000.000 |

The sunscreen has acceptable UVA “blocking” properties and Sun Protection Factor (SPF).

### Example 3

The following composition was prepared in accordance with the method of Example 1.

| | Formula % | Raw Material Type | |
|---|---|---|---|
| A | 45.0 | Illipe Butter | 450.000 |
| | 10.75 | Shea Butter | 107.500 |
| | 5.0 | Moringa Oil | 50.000 |
| | 6.25 | Octocrylene | 62.500 |
| | 3.75 | Butylmethoxydibenzoylmethane | 37.500 |
| B | 12.00 | Calamine Powder | 120.000 |
| | 11.75 | Kaolin | 117.500 |
| | 4.50 | Magnesium Carbonate | 45.000 |
| C | 1.00 | Fragrance | 10.000 |
| | 100.000 | | 1000.000 |

The sunscreen has acceptable UVA “blocking” properties and Sun Protection Factor (SPF).

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A sunscreen in solid form comprising:
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

2. The sunscreen according to claim 1, wherein the sunscreen comprises vegetable oil, vegetable butter or mixtures thereof in a total amount of from 40 to 60% by weight of the sunscreen.

3. The sunscreen according to claim 1, wherein the sunscreen comprises vegetable oil, vegetable butter or mixtures thereof in a total amount of from 60 to 80% by weight of the sunscreen.

4. The sunscreen according to claim 1, wherein the vegetable oil, and vegetable butter are selected from avocado butter, cocoa butter, murumuru butter, cupuacu butter, illipe butter, mango butter, shea butter, sesame oil, rosehip oil, almond oil, raspberry seed oil, moringa oil and mixtures thereof.

5. The sunscreen according to claim 1, wherein the sunscreen comprises vegetable oil, vegetable butter, wax or mixtures thereof in a total amount of from 60 to 80% by weight of the sunscreen.

6. The sunscreen according to claim 1, wherein the sunscreen comprises UV absorbing material in an amount of from 10 to 40% by weight of the sunscreen.

7. The sunscreen according to claim 1, wherein the sunscreen comprises UV absorbing material in an amount of from 10 to 30% by weight of the sunscreen.

8. The sunscreen according to claim 1, wherein the sunscreen comprises UV absorbing material in an amount of from 15 to 25% by weight of the sunscreen.

9. The sunscreen claim 1, wherein the UV absorbing material is selected from octocrylene, octyl methoxycinnamate, butylmethoxydibenzoylmethane, homosalate, ecamsule, and mixtures thereof.

10. The sunscreen according to claim 1, wherein the sunscreen comprises UV reflecting material in a total amount of from 10 to 25% by weight of the sunscreen.

11. The sunscreen according to claim 1, wherein the UV reflecting material is selected from clays, zinc oxide, titanium dioxide and mixtures thereof.

12. The sunscreen according to claim 1, wherein the UV reflecting material comprises at least a clay.

13. The sunscreen according to claim 1, wherein the UV reflecting material comprises at least (i) a clay and (ii) at least one of zinc oxide and titanium dioxide.

14. The sunscreen according to claim 11 wherein the clay is selected from, kaolin, talc, rhassoul mud powder, mica and mixtures thereof.

15. The sunscreen according to claim 1, further comprising one or more fragrances.

16. The sunscreen according to claim 15, wherein the sunscreen comprises fragrance in an amount of no greater than 5% by weight of the sunscreen.

17. The sunscreen according to claim 1, further comprising a component selected from binders, fillers, fruits, vegetables, humectants, dispersants and mixtures thereof.

18. The sunscreen according to claim 1, further comprising a pigment.

19. The sunscreen according to claim 18, wherein the pigment is a UV reflecting material.

20. The sunscreen claim 1, wherein the sunscreen comprises water in an amount of no greater than 5% by weight of the sunscreen.

21. The sunscreen according to claim 1, wherein the sunscreen is free or substantially free of water.

22. The sunscreen according to claim 1, wherein the sunscreen is free or substantially free of preservatives.

23. The sunscreen according to claim 1, wherein the sunscreen is provided free of packaging.

24. A sunscreen in solid form, formulated such that the sunscreen may be washed on to the body.

25. A sunscreen in solid form, formulated such that when a predefined dose of the sunscreen is applied to the body, adequate coverage of the body to a specified UV protective rating is provided.

26. A process for the production of a sunscreen in solid form comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen,
the process comprising the steps of
(a) when the vegetable oil, vegetable butter or mixture thereof is solid, heating the vegetable oil, vegetable butter or mixture thereof to provide a liquid,
(b) mixing the one or more UV absorbing materials, and the one or more UV reflecting material, with the liquid vegetable oil, vegetable butter or mixture thereof,
(c) cooling the mixture to provide the sunscreen in a predefined solid shape.

27. (canceled)

28. A method of applying a predefined dose of sunscreen comprising the step(s) of washing on to the wet skin of a user a sunscreen in solid form and comprising
(i) vegetable oil, vegetable butter or mixture thereof, wherein vegetable oil and vegetable butter are present in a total amount of from 40 to 80% by weight of the solid sunscreen,
(ii) one or more UV absorbing materials, wherein UV absorbing material is present in an amount of from 10 to 55% by weight of the solid sunscreen, and
(iii) one or more UV reflecting materials, wherein UV reflecting material is present in an amount of from 0.5 to 30% by weight of the solid sunscreen.

29. The method according to claim 26, wherein the solid sunscreen is washed on during showering or bathing by the user.
